(19) United States
(12) Patent Application Publication
(10) Pub. No.: US 2023/0346659 A1
(43) Pub. Date: Nov. 2, 2023

(54) ALCOHOL-BASED DISINFECTANT FOAM

(71) Applicant: PROF4SKIN GMBH, Greven (DE)

(72) Inventor: Rolf DANIELS, Rottenburg (DE)

(21) Appl. No.: 18/021,189

(22) PCT Filed: May 18, 2021
(86) PCT No.: PCT/IB2021/054269

§ 371 Date: Feb. 14, 2023

(30) Foreign Application Priority Data
Aug. 14, 2020 (DE) 10 2020 004 965.6

(51) Int. Cl. A61K 8/55; A61K 8/34; A61Q 17/00; A61K 8/04

(52) U.S. Cl. CPC A61K 8/34; A61K 8/046; A61K 8/553; A61Q 17/005; A61K 2800/30; A61K 2800/34

(57) ABSTRACT

The present invention relates to a disinfectant foam based on alcohol for disinfecting the skin. The disinfection foam according to the invention avoids the disturbance of the skin barrier that is usual with alcohol-based disinfectants and does not require silicone surfactants or polyfluorinated chemicals.

### CROSS-REFERENCE TO RELATED APPLICATION

This application is a national phase in the United States of PCT application number PCT/IB2021/054269, filed May 18, 2021, and claiming the benefit of German Application No. 10 2020 004 965.6, filed Aug. 14, 2020, the contents of which are incorporated herein by reference.

### BACKGROUND OF THE INVENTION

The present invention relates to a disinfectant foam based on alcohol for disinfecting the skin. The disinfectant foam according to the invention avoids the disturbance of the skin barrier that is usual with alcohol-based disinfectants and does not require silicone surfactants or polyfluorinated chemicals.

Disinfection of the skin, especially the hands, is essential for many medical purposes. At the latest since the occurrence of the Covid 19 pandemic, regular disinfection of hands in the household has also become necessary. Alcohols, especially ethanol, have proven to be effective for disinfection. However, the use of liquid alcohols for disinfection of the skin has some disadvantages: On the one hand, disinfecting alcohols tend to disrupt the skin barrier, especially drying out the skin. Particularly in the winter months, regular use leads to dry skin, as the body’s own sebum production stops at temperatures below 8° C. However, even distribution on the hand is also problematic. In addition, liquid alcohol tends to drip off the hand. Liquid alcohol on the floor poses a slipping hazard, and dripping alcohol on clothing or shoes causes staining.

From other cosmetic applications, the dosage form of foam has proved successful, as foams are easy to dose and can be evenly distributed on the skin. Attempts have already been made to establish alcohol-based foams as disinfectants. Due to the reduced interfacial tension of alcohol and air compared to water, the production of a stable foam is a challenge.

EP 1858323 B1, for example, describes an alcohol-based foam produced by means of silicone-containing surfactants. However, such silicone-containing surfactants pose an environmental problem because they are only very slightly biodegradable.

WO 00/47183 A describes alcohol-based gels for skin disinfection, where the gels are stabilized by silicone-containing polymers and by polyfluorinated chemicals (e.g., perfluoropolypropyl ether). Polyfluorinated chemicals, too, are of ecological concern.

US 9023374 B2 (based on WO 2011/039630 A1) describes alcohol-based compositions for disinfection containing docusate sodium (sodium dioctyl sulfosuccinate). However, the compositions are not suitable for the formation of stable foams (see examples below).

US 2016/0250111A1 describes alcohol-based hand sanitizers, but not foams.

There is therefore a need for improved alcohol-based disinfection foams that do not use silicone surfactants or polyfluorinated chemicals.

Surprisingly, it was found that such disinfection foams could be prepared without the use of silicone surfactants and without polyfluorinated chemicals. The formulations according to the invention contain hydrogenated phospholipids for foam stabilization, in particular phospholipon 80H and/or phospholipon 90H.

The hydrogenated phospholipids are preferably obtained from phospholipids of natural origin. Phospholipids from plants, such as soybean lecithin, are particularly suitable. The phospholipids can be characterized by the phosphatidylcholine content, which is the main ingredient of phospholipids. The hydrogenated phospholipids used according to the invention are prepared from these native phospholipids by hydrogenation. Phospholipids, which are purified and have a high phosphatidylcholine content, prove to be particularly favorable.

For the hydrogenated phospholipids, the phosphatidylcholine content is at least about 70% by weight.

The hydrogenated phospholipid used according to the invention may also be a mixture of different hydrogenated phospholipids and, in particular, a mixture of hydrogenated phosphatidylcholine and hydrogenated lysophosphatidylcholine. In such a mixture, the weight ratio of hydrogenated phosphatidylcholine to hydrogenated lysophosphatidylcholine should be between 97:3 and 40:60.

Known phospholipids that fulfill these properties are available, for example, from Phospholipid GmbH (Cologne, Germany) under the designations Phospholipon 80H and Phospholipon 90H. Phospholipon 80H comprises about 76% hydrogenated phosphatidylcholine and about 3% hydrogenated lysophosphatidylcholine, Phospholipon 90H, a hydrogenated phosphatidylcholine, comprises at least 90% hydrogenated phosphatidylcholine and a maximum of 4% hydrogenated lysophosphatidylcholine. However, hydrogenated phospholipids from other manufacturers can also be used for the preparations according to the invention.

The proportion of hydrogenated phospholipids in the foamable preparation is 0.5 to 5% by weight. A mixture of different hydrogenated phospholipids is also suitable. These hydrogenated phospholipids are added to an aqueous alcohol solution at a temperature between 40 and 60° C. with stirring.

Suitable alcohols are in particular ethanol, n-propanol, and isopropanol, of which ethanol is particularly preferred. The alcohol content of the solution is at least 50% by weight, alcoholic solutions of 60 to 75% by weight are particularly preferred. Mixtures of the alcohols mentioned are also suitable.

### SUMMARY OF THE INVENTION

The invention, therefore, relates to foamable alcohol-based compositions for skin disinfection, which are free of silicone surfactants and free of polyfluorinated chemicals, comprising
a) ethanol, n-propanol, and/or i-propanol in an aqueous solution with an alcohol content of more than 50%, and
b) one or more hydrogenated phospholipids in a proportion of 0.5-5% by weight with respect to the total amount of the liquid, according to claim 1.

Further advantageous embodiments are the subject of the subclaims.

### DETAILED DESCRIPTION OF THE INVENTION

The preparation described can be further stabilized by a proportion of ethanol-soluble cellulose ethers, such as Tylose PSO, typically less than 1% by weight.

Furthermore, the preparation may contain up to 10% by weight glycerol and up to 6% by weight hydrogen peroxide.

An addition of 1-10% by weight (preferably 2.5-7.5% by weight) urea has proved to be particularly advantageous: Foams according to the invention retain their foam structure and advantageous disinfection properties even with the addition of urea. At the same time, urea-containing foams reduce occasional skin sensitivities to alcohols.

A number of compositions according to the invention are shown by way of example in Tables 1 to 12. The compositions shown in Tables 1 to 12 are first mixed (typically by stirring the hydrogenated phospholipids into the aqueous alcohol solution at 40 to 60° C.) and then filled into suitable pressure vessels made of glass, aluminum, tinplate, or plastic, closed with a foam valve, e.g., a precision valve, and then preferably pressurized with about 2 to 10% by weight of a gas or a gas mixture (e.g., propane/butane). An equilibrium pressure of 1-5 bar is achieved at room temperature by the gas or gas mixture. By actuating the spray head, the desired quantities of foam can be extracted by a commercially available foam head, e.g., model “Fiji” from Precision Dispensing Solutions Europe.

The products according to the invention are suitable for effectively disinfecting the skin, in particular the hand. For this purpose, an approximately walnut-sized amount of foam is taken from a dispenser and evenly distributed on the desired part of the body, in particular the hand. The foam is characterized by excellent adhesion to the skin. The adhesion also prevents the dripping of alcohol. A protective screen is formed on the skin, which is hardly soluble in water and acts as a protective barrier. Disinfection can be performed regularly without drying out or otherwise irritating the skin. The foam according to the invention is bactericidal, virucidal, levurocidal, and myobactericidal.

The compositions according to the invention may contain the emulsifiers and emulsion stabilizers known from similar foams, care agents, viscosity regulators, surfactants, and pH buffers. The addition of film-forming polymers is also possible. In particular, polyvinyl alcohol, PVP, copovidones, cellulose ethers, polyvinyl-co-polymers, and polymethacrylate-co-polymers should be mentioned here. The sum of these optional additives should not exceed 5%.

The addition of fragrances is possible, but is not recommended to avoid allergy risks.

By way of example, some compositions are disclosed in the following tables, without these being intended to constitute a limitation. The person skilled in the art can make numerous variations and further developments without having to be inventive.

Unless otherwise stated, all% figures in this publication are% by weight.

### EXAMPLES

TABLE 1

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| Ethanol 70% | Ad 100% | Ad 100% | Ad 100% | | | |
| Ethanol 60% | | | | Ad 100% | Ad 100% | Ad 100% |

Charged with 2 - 10% propane-butane mixture with an equilibrium pressure of 1 - 5 bar (i.e.: 2 to 10 g of propane-butane mixture are added to 100 g of the solutions with the above-mentioned compositions).

TABLE 2

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| Glycerol | 5% | 5% | 5% | 5% | 5% | 5% |
| Ethanol 70% | Ad 100% | Ad 100% | Ad 100% | | | |
| Ethanol 60% | | | | Ad 100% | Ad 100% | Ad 100% |

Charged with 2 - 10% propane-butane mixture with an equilibrium pressure of 1 - 5 bar.

TABLE 3

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| Tylose PSO | 0.5% | 0.5% | 0.5% | 0.5% | 0.5% | 0.5% |
| Ethanol 70% | Ad 100% | Ad 100% | Ad 100% | | | |
| Ethanol 60% | | | | Ad 100% | Ad 100% | Ad 100% |

Charged with 2 - 10% propane-butane mixture with an equilibriumpressure of 1 - 5 bar.

TABLE 4

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| Glycerol | 5% | 5% | 5% | 5% | 5% | 5% |
| Tylose PSO | 0.5% | 0.5% | 0.5% | 0.5% | 0.5% | 0.5% |
| Ethanol 70% | Ad 100% | Ad 100% | Ad 100% | Ad 100% | Ad 100% | Ad 100% |

Charged with 2 - 10% propane-butane mixture with an equilibrium pressure of 1 - 5 bar.

TABLE 5

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| i-Propanol 70% | Ad 100% | Ad 100% | Ad 100% | | | |
| i-Propanol 60% | | | | Ad 100% | Ad 100% | Ad 100% |

Charged with 2 - 10% propane-butane mixture with an equilibrium pressure of 1 - 5 bar.

TABLE 6

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| n-Propanol 70% | Ad 100% | Ad 100% | Ad 100% | | | |
| n-Propanol 60% | | | | Ad 100% | Ad 100% | Ad 100% |

Charged with 2 - 10% propane-butane mixture with an equilibrium pressure of 1 - 5 bar.

TABLE 7

| | | | | | | |
|---|---|---|---|---|---|---|
| Constituent | A | B | C | D | E | F |
| Phospholipon 80H | 0.5-2, 5% | | 0.5-2, 5% | 0.5-5% | | 0.5-2, 5% |
| Phospholipon 90H | 0.5-2, 5% | 0.5-5% | | 0.5-2, 5% | 0.5-5% | |
| Other hydrated phospholipids | | 0.5-2, 5% | 0.5-5% | | 0.5-2, 5% | 0.5-5% |
| Ethanol 70% | Ad 100% | Ad 100% | Ad 100% | | | |
| i-Propanol 60% | | | | Ad 100% | Ad 100% | Ad 100% |

Charged with 2 - 10% propane-butane mixture with an equilibrium pressure of 5 bar.

TABLE 9

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| Glycerol | 6% | 8% | 10% | 6% | 8% | 10% |
| Ethanol 80% | Ad 100% | Ad 100% | Ad 100% | | | |
| Ethanol 75% | | | | Ad 100% | Ad 100% | Ad 100% |

Charged with 2 - 10% propane-butane mixture with an equilibrium pressure of 1 - 5 bar.

TABLE 10

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| Glycerol | 10% | 8% | 6% | 10% | 8% | 6% |
| Tylose PSO | 0.5% | 0.5% | 0.5% | 0.5% | 0.5% | 0.5% |
| Ethanol 75% | Ad 100% | Ad 100% | Ad 100% | Ad 100% | Ad 100% | Ad 100% |

Charged with 2 - 10% propane-butane mixture with an equilibrium pressure of 1 - 5 bar.

TABLE 11

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| Glycerol | 10% | 8% | 6% | 10% | 8% | 6% |
| Urea | 2.5% | 5% | 7.5% | 7.5% | 5% | 2.5% |
| Ethanol 75% | Ad 100% | Ad 100% | Ad 100% | Ad 100% | Ad 100% | Ad 100% |

Charged with 2 - 10% propane-butane mixture with an equilibrium pressure of 1 - 5 bar.

TABLE 12

| | | | | | | |
|---|---|---|---|---|---|---|
| Constituent | A | B | C | D | E | F |
| Phospholipon 80H | 0.5-5% | | | 0.5-5% | | |
| Phospholipon 90H | | 0.5-5% | | | 0.5-5% | |
| Other hydrated phospholipids | | | 0.5-5% | | | 0.5-5% |
| Urea | 2.5% | 5% | 7.5% | 7.5% | 5% | 2.5% |
| Ethanol 80% | Ad 100% | Ad 100% | Ad 100% | Ad 100% | Ad 100% | Ad 100% |

Charged with 2 - 10% propane-butane mixture with an equilibrium pressure of 1 - 5 bar.

### Comparative Example

TABLE 13

| | | | | | | |
|---|---|---|---|---|---|---|
| Constituent | A | B | C | D | E | F |
| Phospholipon 90H | 6.0 | 5.0 | | | 6.0 | 6.0 |
| Phosphatidylcholine | | | 6.0 | 6.0 | | |
| Glycerol | | 1.0 | | 2.0 | | 2.0 |
| Docusate sodium | | | | 0.2 | 0.2 | 0.2 |
| Menthol | | | | 0.2 | | |
| Geraniol | | | | 0.2 | | |
| Vitamin E acetate | | | | 0.6 | | |
| Ethanol | 65.0 | 65.0 | 65.0 | 65.0 | 65.0 | 65.0 |
| Water | Ad 100% | Ad 100% | Ad 100% | Ad 100% | Ad 100% | Ad 100% |

The compositions according to Table 13 (A-F) are each mixed under stirring at 40-60° C., poured into a pressure vessel, and charged with approx. 5% propane-butane mixture. The pressure vessel is fitted with a commercially available spray head for foams. The composition of Table 13C corresponds to Example 34 of US 9,023,374 B2.

At room temperature, an attempt is made to extract an approx. walnut-sized quantity of a foam by actuating the foam head.

Result: The compositions according to Table 13A and B give excellent, fine-pored foams. The extracted foam remains stable for several minutes. The foam spreads excellently on the skin without dripping.

The compositions according to Table 13C to D give very coarse-pored foams, which already have a certain liquid content on removal. The foam content dissipates immediately after removal. After considerably less than one minute, there is practically no foam left. When an attempt is made to spread the foam on the hand, liquid drips off the hand.

1. A foamable alcohol-based composition for disinfecting the skin, free from silicone surfactants and free from polyfluorinated chemicals, containing
a) ethanol, n-propanol, and/or i-propanol in an aqueous solution with an alcohol content of more than 50%, and
b) one or more hydrogenated phospholipids in a proportion of 0.5-5% by weight with respect to the total amount of the liquid.

2. The foamable alcohol-based composition for disinfecting the skin according to claim 1, wherein the hydrogenated phospholipid is selected from phospholipon 80H and/or phospholipon 90H.

3. The foamable alcohol-based composition for disinfecting the skin according to claim 1, wherein the aqueous solution of ethanol, n-propanol, and/or i-propanol has an alcohol content of 60-75% by weight.

4. The foamable alcohol-based composition for disinfecting the skin according to claim 1, wherein the alcohol is ethanol.

5. The foamable alcohol-based composition for disinfecting the skin according to claim 1, containing up to 10% by weight glycerol.

6. The foamable alcohol-based composition for disinfecting the skin according to claim 1, containing up to 1% by weight cellulose ether.

7. The foamable alcohol-based composition for disinfecting the skin according to claim 1, optionally containing 1 to 10% by weight urea.

8. The foamable alcohol-based composition for disinfecting the skin according to claim 1 in a pressure vessel with a spray head for foams, charged with 2-10% by weight of a gas or gas mixture with an equilibrium pressure of 1-5 bar.

9. An alcohol-based disinfectant foam for disinfecting the skin, obtainable by extracting a foamable alcohol-based composition according to claim 1, charged with 2-10% by weight of a gas or gas mixture having an equilibrium pressure of 1-5 bar, from a pressure vessel having a spray head for foams.
